(19) United States
(12) Patent Application Publication
(10) Pub. No.: US 2017/0173230 A1
(43) Pub. Date: Jun. 22, 2017

(54) WOUND THERAPY

(71) Applicant: Shree Devi Visaria, Houston, TX (US)

(72) Inventor: Shree Devi Visaria, Houston, TX (US)

(21) Appl. No.: 15/453,658

(22) Filed: Mar. 8, 2017

Related U.S. Application Data
(60) Provisional application No. 62/375,333, filed on Aug. 15, 2016.

(51) Int. Cl. A61M 1/00

(52) U.S. Cl. CPC A61M 1/0088; A61M 1/0025; A61M 1/0072; A61M 2210/1021

(57) ABSTRACT

A wound drain includes a drain tube configured to drain fluid from an interior cavity of a wound, a fluid wicking bandage configured to wick fluid from a surface of the wound, and a suction flange configured to pull fluid from the drain tube and the fluid wicking bandage into a suction flange tube. The suction flange tube is configured to be connected to a vacuum source for draining fluid from the wound.

### CROSS REFERENCE TO RELATED APPLICATIONS

The present application is a non-provisional application that claims the benefit of, and the priority from, U.S. Provisional Patent Application No. 62/375,333 having a title of “Wound Therapy,” filed Aug. 15, 2016, which is incorporated entirely herein.

### FIELD

The present invention relates, generally, to systems and methods usable for managing and providing therapy to wounds. Particularly, wounds may result from surgical incisions into a patient's abdominal cavity, and drain(s) may be needed to siphon fluid from within and around the incision, in addition to topically protecting and draining the fluid from the surface of the skin.

### BACKGROUND

Surgeons and other physicians providing post-op care to surgery patients have a need to quickly, efficiently, and cleanly drain the fluid formed within the body and around the incision to promote the healing process. The fluid accumulation may be particularly problematic in patients with comorbid conditions such as obesity, diabetes, immunosuppression, and/or high tension wounds. To remove this accumulation of fluid, these physicians have used forms of deep tissue drains and bandages for minimizing the problems in the healing process caused by the accumulation of the fluid.

In some surgery patients, fluid can develop below the surface of an incision, causing a seroma to form. This fluid develops due to leakage of interstitial fluid that may drain for days or weeks after the surgery is completed, and during the healing process. The tissue below the incision tends to seep fluid that needs to be directed away from the general incision area and out of the body.

The problem of removing this accumulation of fluid is particularly pronounced in patients that have a significant amount of tissue (e.g., adipose tissue, etc.) below the skin surface. For these patients, the amount of serous fluid draining into the wound may exceed the body's natural lymphatic drainage abilities, or draining limits of the existing draining systems and techniques. When this happens, the skin above the wound may become saturated by fluid that drains through the wound to the surface of the skin. That is, some serous fluid (or other fluids) may seep passed the patient's sutures, staples, and/or other wound closure treatments, to stagnate on the patient's skin. As such, this extra, stagnate fluid can cause severe discomfort to the patient, maceration of the patient's skin, and/or a significantly higher risk of infection to the patient. Therefore, a need exists for apparatus and methods usable to remove fluid, including an accumulation of fluid, within the body in a sufficient amount (e.g., from multiple locations) that leaves the patient's skin, surrounding the wound, dry and sterile (e.g., free from bacterial growth and infection).

In addition, a need exists for a wound drain that is straightforward and easy to install. In some instances, surgeons may default to only applying topical bandages to absorb the fluid leaking from around the surface of the wound and the patient's skin. The surgeon may have multiple patients and/or appointments to attend to in a given shift, and the extra time it takes to install a drain may influence the surgeon to merely apply a bandage. Furthermore, as described above, a seroma may form beneath the closed wound. Seromas may contribute to wound infection and/or wound dehiscence, which may not present for up several weeks after the surgery. Therefore, a need exists for a deep tissue drain that is just as easy and quick to apply as a conventional topical bandage.

The present embodiments meet all of these needs.

### SUMMARY

The embodiments disclosed herein may include a bandage kit for draining a wound of a surgical procedure performed on a patient. For example, the bandage kit may include a first drain tube configured to drain fluid from a first location within an interior cavity of the wound, a second drain tube configured to drain fluid from a second location within the interior cavity of the wound, and a connecting tube configured to drain the fluid from the first drain tube and the second drain tube. A length of the connecting tube may be selected according to the surgical procedure. The bandage kit may also include a fluid wicking covering configured to wick surface fluid from a surface of the wound. The connecting tube may be located within the fluid wicking covering and configured to be implemented concurrently with the fluid wicking covering. The bandage kit may also include a vacuum source connected directly to the fluid wicking covering, and configured to drain the fluid from the connecting tube.

Further embodiments may also include a flange configured to pull fluid from the drain tube and the fluid wicking covering. The flange in the kit may be configured to lie flat against the patient, and the vacuum source may include an electronic pump, a vacuum bulb, or combinations thereof. The first drain tube, second drain tube, or combination thereof, may include a substantially straight semi-stiff drain tube.

In certain embodiments, the first drain tube, second drain tube, or both, are perpendicular to the connecting tube. And, the first drain tube, second drain tube, or combination thereof may be configured to slide into a gap in a sealing of the wound. The bandage kit may include a measuring device configured to match to the surgical procedure, wherein the measuring device indicates proper location of the gap for first drain tube, second drain tube, or combinations thereof. In certain embodiments, the fluid wicking covering may be configured to absorb at least five days of surface fluid at the surface of the wound.

Disclosed embodiments also include a method of placing a bandage kit over a wound of a surgical procedure performed on a patient's body. The method may include sealing a portion of the wound while leaving a gap configured to fit a drain tube of the bandage kit, inserting the drain tube into the gap, wherein the drain tube is attached to a connecting tube within a fluid wicking covering, placing the fluid wicking covering on a skin surface to cover the wound, and connecting the suction flange to a vacuum source.

In certain embodiments, the method may include measuring, with a measuring device specific to the surgical procedure, a proper location for the gap, and/or cutting the wound into the patient's body to perform a surgery. In certain embodiments of the method, the surgery may include operating on the patient′ abdominal cavity.

The method may also include leaving a second gap configured to fit a second drain tube of the bandage kit, and simultaneously inserting the drain tube and the second drain tube into the gap and the second gap. The drain tube and the second drain tube can both be attached to the connecting tube. The method may include trimming the drain tube to a length based on a measured depth of the wound.

Disclosed embodiments also include a method of placing a bandage kit over a wound of a surgical procedure performed on a patient. The method may include cutting a portion of a sealing of the wound to create a gap configured to fit a drain tube of the bandage kit, inserting the drain tube into the gap, wherein the drain tube is attached to a connecting tube within a fluid wicking covering, placing the fluid wicking covering on a skin surface to cover the wound, securing a seal over the fluid wicking covering, and connecting the suction flange to a vacuum source.

### BRIEF DESCRIPTION OF THE DRAWINGS

FIG. 1 depicts a perspective view of a patient having a surgical wound from an abdominal-cavity operation.

FIG. 2 illustrates a perspective view of a patient with an embodiment of a negative pressure wound drain placed within the wound.

FIG. 3 illustrates a cross-sectional side view of an embodiment of the negative pressure wound drain that has been placed within the wound of the patient.

FIG. 4 is a perspective view of patient body with a closed wound from an abdominal operation that is ready for application of bandage kit.

### DETAILED DESCRIPTION OF THE EMBODIMENTS

Before explaining selected embodiments of the present invention in detail, it is to be understood that the present invention is not limited to the particular embodiments described herein and that the present invention can be practiced or carried out in various ways.

The present invention relates, generally, to a system usable to provide negative pressure wound therapy for primary skin closures and deep tissue drains. The disclosed embodiments are particularly beneficial for patients with conditions that may impair wound healing. Such conditions may include obesity, diabetes, age, steroid use, immunosuppression, and impaired oxygenation. The disclosed embodiments may be used with clean, primary incision closures for any type of laparotomy, large suprafascial procedures (e.g., abdominoplasty), and large, deep soft tissue incisions.

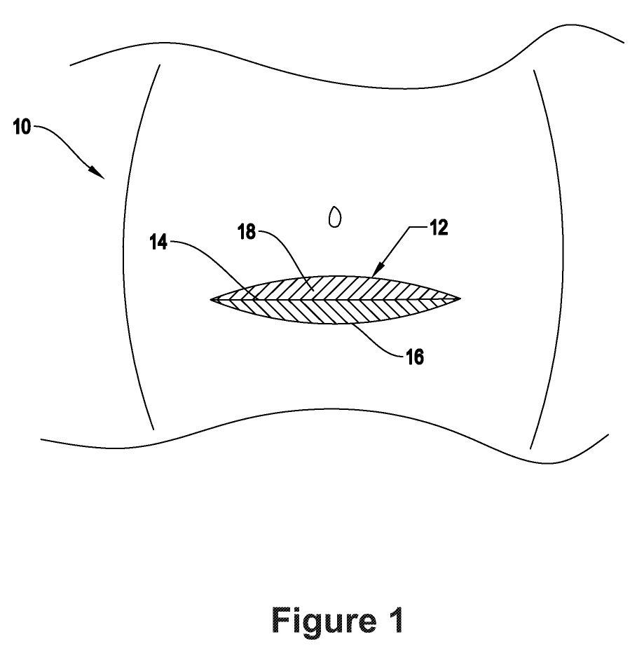

FIG. 1 depicts a perspective view of a patient body 10 having a wound 12 (e.g., surgical wound, surgical incision) from an abdominal-cavity operation. The disclosed embodiments below may be addressed, in particular, to operations involving abdominal wounds, but the embodiments may be understood to be used in any deep-tissue procedure in any location, or orientation, on the patient's body 10. Additionally, while embodiments of the disclosed invention may be used in subfascial procedures, the description will focus on draining operations after a fascia 14 is closed, or has not been cut. FIG. 1 illustrates the wound 12 through a skin layer 16, a subcutaneous layer 18, to the fascia 14. As outlined in the background above, the surgeon or doctor has typically finished the procedure at this point and is, at the point illustrated in FIG. 1, ready for cleaning and sealing the wound 12. In some patients (but not every patient), the subcutaneous layer 18 can produce fluid after the surgery, and all through the healing process. The surgeon or doctor may evaluate the need for a patient to use a drain, taking into account the discomfort for the patient to care for the drain (e.g., empty a reservoir of the fluid). As shown and described, the disclosed embodiments of the bandage kit for applying a negative pressure therapy to the wound or primary closed incision, will work for many or all different types of surgical incisions while decreasing discomfort in the patient and increasing ease of application by the surgeon or doctor performing the procedure.

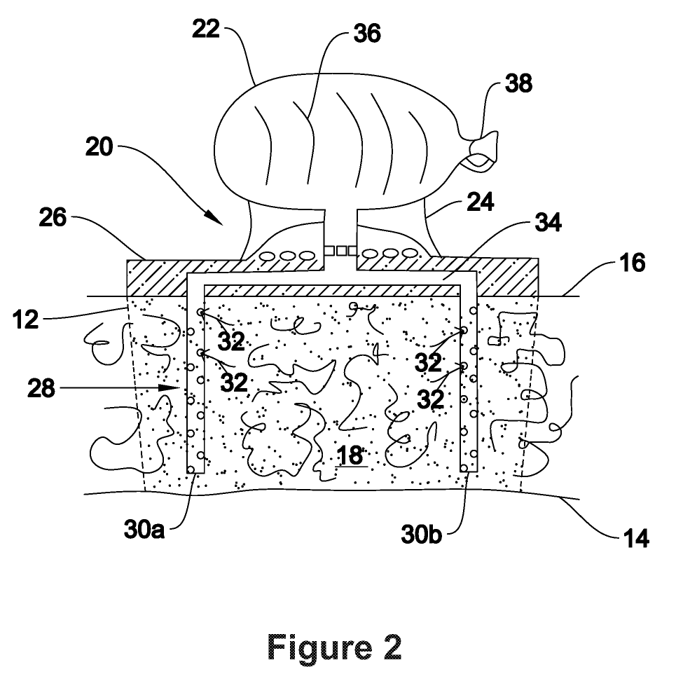

FIG. 2 illustrates a cross-sectional view of the patient's body 10 with a bandage kit 20 applied to the wound 12 (e.g., surgical incision). The method of installation will be described below, but FIG. 2 shows the bandage kit 20 as it would be applied to the wound 12. The bandage kit 20, as shown, includes a vacuum source 22, a connecting flange 24, and a fluid wicking covering 26 on the surface 16 of the wound 12. Internal to the wound 12, the bandage kit 20 includes drain tubes 28. After application, the bandage kit 20 can be secured to the patient's body 10 through adhesion of the fluid wicking covering 26 and may adjust only slightly as the patient moves. In certain embodiments, the external components (e.g., vacuum source 22, connecting flange 24, and fluid wicking covering 26) may be configured to lie flat under the patient's clothing, while still absorbing and suctioning fluid. Lying flat under the patient's clothing may be that the flange lies against the patient within 2.54 cm (1 inch), 1.27 cm (0.5 inches), or within 0.64 cm (0.25 inches) of the patient's skin. This would enable the patient a broader range of movement over the several days or weeks of recovery. In other embodiments, the fluid may be carried to a remote vacuum source 22 that includes a larger reservoir for fluid accumulation.

The internal components of the bandage kit 20 may include the drain tubes 28. The drain tubes 28, in certain embodiments, may be flat to enable the fluid to be pulled from the wound 12 over a broad area. In certain other embodiments, the drain tubes 28 may be round, oblong, oval, or other shapes depending on the size, shape, or other characteristics of the wound 12. In the illustrated embodiment, the drain tubes 28 include a first drain tube 30a and a second drain tube 30b that are round and fenestrated along a length. As shown, the first drain tube 30a and the second drain tube 30b include holes 32 that receive fluid from within the wound 12. The holes 32 may vary in size and shape to tailor the fluid recovery to specific locations within the wound 12. For example, some embodiments of the bandage kit 20 may include a difference between the size of the holes 32 of the first drain 30a and the second drain 30b. The first drain 30a may have holes 32 that are bigger (e.g., suctioning more fluid) than the second drain 30b, for wounds 12 that have a tendency to produce fluid at a non-uniform amount depending on location. For similar reasons, the drain tubes 28 may, in some embodiments, have holes 32 that are larger at the bottom to ensure that fluid does not pool near the fascia 14.

The first drain 30a and the second drain 30b may also differ in the material of manufacture. The drain tubes 28 may be constructed of many different plastic, polymer, natural, or synthetic materials, or materials coated in one of these materials to ensure that the drain tubes 28 are unreactive to the patient's body 10. For example, the drain tubes 28 may be manufactured from flexible material that is able to adjust and move with the patient to ensure comfort and mobility. In other embodiments, the drain tubes 28 may be manufactured from a sturdier material to ensure a quick and easy insertion as explained in detail below. The first drain 30a and the second drain 30b may be independently manufactured so that each has a unique material of combination of materials/coatings. Unique combinations of materials and other characteristics customized for each bandage kit 20 may enable a doctor/surgeon to quickly select a bandage kit 20 that matches the procedure, the wound 12, and the patient's body 10. Furthermore, the drain tubes 28 may be trimmed immediately before insertion to increase the customization to the patient's body 10.

After the drain tubes 28 direct fluid from the wound 12, the fluid is drawn through a connecting tube 34 and into a vacuum source 22. The connecting tube 34, in the illustrated embodiment, is straight and connected to the drain tubes 28 at a right angle, such that the drain tubes 28 are perpendicular to the fluid wicking covering 26 and the skin surface 16. In other embodiments, the connecting tube 34 may be curved so that the drain tubes 28 connect to the connecting tube 34 at a different angle while still be inserted perpendicularly into the patient's body 10. The connecting tube 34 may also define a length between the drain tubes 28 that is specifically matched to the procedure. The length between the drain tubes 28 may be selected so that the fluid from the wound is collected at the highest efficiency. For example, if a particular procedure is conducted with a surgical wound that is 10 cm (3.94 inches) long, the wound drains 28 may collect fluid most efficiently when placed at 3 cm (1.18 inches) and 7 cm (2.76 inches) from one end of the wound. The bandage kit 20 that a surgeon would pick for that procedure, therefore, would have a length of 4 cm (1.57 inches) between the drain tubes 28.

The vacuum source 22 may include mechanical, electronic, or other vacuum sources to maintain a pressure differential. In an embodiment, the pressure differential may be between 0.0 mm Hg and 170 mm Hg. Furthermore, the vacuum source 22 may be located adjacent to the patient's body 10, or may be remotely located and connected to the remaining components of the bandage kit 20 by a joining tube. The vacuum source 22 may include a bulb-style vacuum with resilience features 36 that return the bulb to an original expanded shape after a user has squeezed the bulb vacuum source. The bulb may be squeezed while a spout 38 is open, allowing the fluid to exit without adding pressure to the wound 12. In the illustrated embodiment, the resilience features 36 are lines of plastic that reinforce the bulbs tendency to return to a rounded shape. The spout 38 is closed during evacuation of the wound 12 and as the bulb returns to the rounded shape, it maintains the pressure differential until it is filled with fluid.

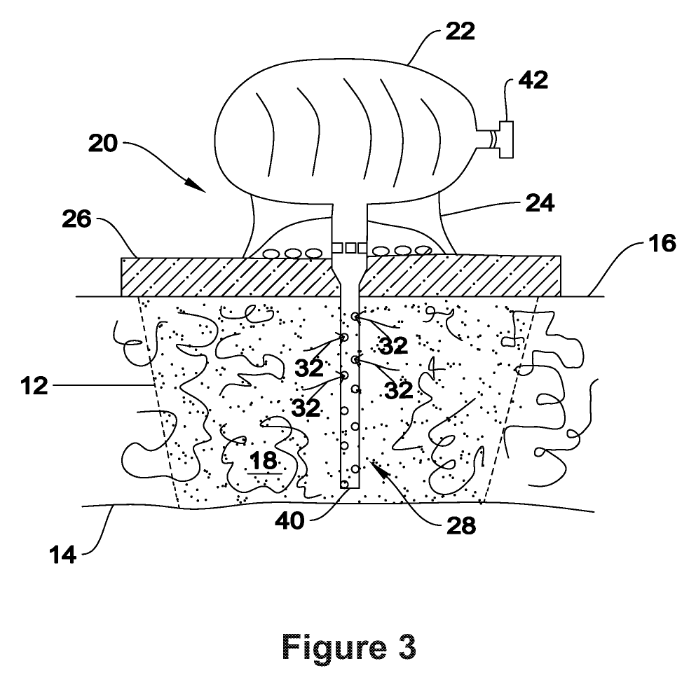

FIG. 3 illustrates a cross-sectional side view of an embodiment of the bandage kit 20 that has been placed within the wound 12 of the patient's body 10. The illustrated embodiment includes a single drain tube 40, as illustrated, has been inserted into the wound 12 at a deep location near the fascia 14. The bandage kit 20 may have more drain tubes 28 depending on the length, depth, and extent of the wound 12. Furthermore, the bandage kit 20 with a single drain tube 40 may be individually placed in multiple locations of the wound 12 for quick installation of fluid removal. For example, in large wounds of non-typical procedures, a bandage kit 20 with multiple drain tubes 28 may not provide a sufficiently customized fluid drainage, requiring instead drains that are individually inserted. To insert bandage kits 20 with single drain tubes 40, the fluid wicking covering 26 may be trimmed so that each bandage kit 20 may be placed right next to adjacent bandage kits 20. Additionally, the fluid wicking coverings 26 may overlap, in certain embodiments, the enable the drain tubes 28 to be inserted close to one another.

To further enable insertion of multiple single-drain tube 40 bandage kits 20, the vacuum source 22 may include a tube connector 42 that attaches to an extending tube. Several extending tubes may convey fluid from several vacuum sources 22 into a single larger reservoir. The larger reservoir enables smaller individual vacuum sources 22, shrinking the overall space of the bandage kit 20. Smaller bandage kits 20 may be placed closer together to increase the fluid suction for a given area.

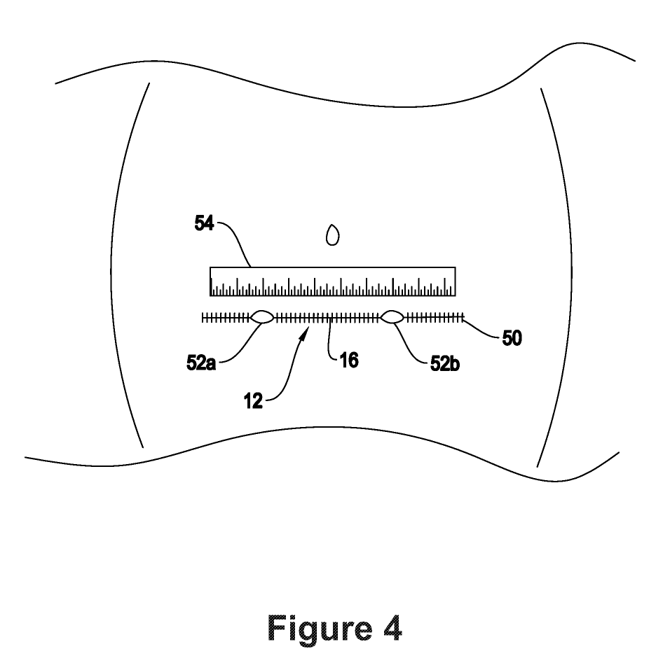

FIG. 4 depicts a perspective view of a patient body 10 having a surgical wound 12 from an abdominal-cavity operation that is ready for insertion of a bandage kit 20. The patient body 10 shows a portion of the wound 12 has been sealed with staples, sutures, or other closure methods 50 while leaving a first gap 52a and a second gap 52b. The gaps 52a, 52b are sized to fit the drain tubes 28 described in detail above. The proper location of the gaps 52a, 52b may be determined by using a measuring device 54 specific to the surgical procedure that has been performed on the patient's body 10. The measuring device 54 may accompany the bandage kit 20 (i.e., with the packaging) so that the doctor/surgeon does not have to memorize, measure, and/or mark the patient during the procedure. The measuring device 54 may specify the location and the size of the gaps 52a, 52b so that the doctor/surgeon knows where to place the sealing structures 50. Additionally or alternatively to leaving the gaps 52a, 52b, the bandage kit 20 may be placed by first entirely sealing the wound 12. The gaps 52a, 52b are then cut into the sealing structures 50 and/or the sealing structures 50 are removed to create the gaps 52a, 52b.

Once the gaps 52a, 52b are created in the sealing of the wound 12, the drain tubes 28 (shown in FIG. 3) may be inserted into the gaps 52a, 52b. As described above, the drain tubes 28 may be trimmed to a certain length to ensure proper suction of the fluid from the wound 12. The drain tube 28 may include semi-stiff tubes that maintain a straight shape with only a small amount of bending or deforming. In certain embodiments, the drain tubes 28 may include a softer material so that the drain tubes 28 do not put an uncomfortable amount of pressure or poking into the patient's body 10. For softer drain tubes 28, an insertion tool may be used to make sure that the drain tubes 28 insert fully into the gaps 52a, 52b without bending.

After the drain tubes 28 are inserted into the gaps 52a, 52b, the fluid wicking covering 26 (shown in FIG. 3) is placed down over the wound 12 and the sealing structure(s) 50. The fluid wicking covering 26, in certain embodiments, contains the connecting tube 34 (shown in FIG. 2) that keeps the drain tubes 28 at the specified distance apart. The fluid wicking covering 26 is also matched to the procedure, such that the wound 12 is covered completely when the drain tubes 28 are measured correctly and inserted into the gaps 52a, 52b. Since the connecting tube 34 and the drain tubes 28 are directly attached within the fluid wicking covering 26, placing of the bandage kit 20 is basically finished. The doctor/surgeon may connect the vacuum source 22 to the connecting flange, check for proper sealing around the fluid wicking covering, and be finished.

While various embodiments of the present invention have been described with emphasis, it should be understood that within the scope of the appended claims, the present invention might be practiced other than as specifically described herein.

1. A bandage kit for draining a wound of a surgical procedure performed on a patient, comprising:
a first drain tube configured to drain fluid from a first location within an interior cavity of the wound;
a second drain tube configured to drain fluid from a second location within the interior cavity of the wound;
a connecting tube configured to drain the fluid from the first drain tube and the second drain tube, wherein a length of the connecting tube is selected according to the surgical procedure;
a fluid wicking covering configured to wick surface fluid from a surface of the wound, wherein the connecting tube is located within the fluid wicking covering and configured to be implemented concurrently with the fluid wicking covering; and
a vacuum source connected directly to the fluid wicking covering, and configured to drain the fluid from the connecting tube.

2. The bandage kit of claim 1, comprising a flange configured to pull fluid from the first drain tube, the second drain tube, and the fluid wicking covering.

3. The bandage kit of claim 2, wherein the flange is configured to lie flat against the patient.

4. The bandage kit of claim 1, wherein the vacuum source comprises an electronic pump, a vacuum bulb, or combinations thereof.

5. The bandage kit of claim 1, wherein the first drain tube, the second drain tube, or combinations thereof, comprises a substantially straight semi-stiff drain tube.

6. The bandage kit of claim 1, wherein the first drain tube, the second drain tube, or combinations thereof, are perpendicular to the connecting tube.

7. The bandage kit of claim 1, wherein the first drain tube, the second drain tube, or combinations thereof is configured to slide into a gap in a sealing of the wound.

8. The bandage kit of claim 7, further comprising a measuring device configured to match to the surgical procedure, wherein the measuring device indicates proper location of the gap for the first drain tube, the second drain tube, or combinations thereof.

9. The bandage kit of claim 1, wherein the fluid wicking covering is configured to absorb at least five days of surface fluid at the surface of the wound.

10. A method of placing a bandage kit over a wound of a surgical procedure performed on a patient's body, comprising:
sealing a portion of the wound while leaving a gap configured to fit a drain tube of the bandage kit;
inserting the drain tube into the gap, wherein the drain tube is attached to a connecting tube within a fluid wicking covering;
placing the fluid wicking covering on a skin surface to cover the wound; and
connecting the suction flange to a vacuum source.

11. The method of claim 10, further comprising measuring, with a measuring device specific to the surgical procedure, a proper location for the gap.

12. The method of claim 10, further comprising cutting the wound into the patient's body to perform a surgery.

13. The method of claim 12, wherein the surgery comprises operating on the abdominal cavity of the patient.

14. The method of claim 10, further comprising leaving a second gap configured to fit a second drain tube of the bandage kit, and simultaneously inserting the drain tube and the second drain tube into the gap and the second gap, respectively, wherein the drain tube and the second drain tube are both attached to the connecting tube.

15. The method of claim 10, further comprising trimming the drain tube to a length based on a measured depth of the wound.

16. A method of placing a bandage kit over a wound of a surgical procedure performed on a patient, comprising:
cutting a portion of a sealing of the wound to create a gap configured to fit a drain tube of the bandage kit;
inserting the drain tube into the gap, wherein the drain tube is attached to a connecting tube within a fluid wicking covering;
placing the fluid wicking covering on a skin surface of the patient to cover the wound;
securing a seal over the fluid wicking covering; and
connecting the suction flange to a vacuum source.

17. The method of claim 16, further comprising measuring, with a measuring device specific to the surgical procedure, a proper location before cutting to create the gap.

18. The method of claim 16, further comprising cutting the wound into the patient's body to perform a surgery.

19. The method of claim 18, wherein the surgery comprises operating on the patient' abdominal cavity of the patient.

20. The method of claim 16, further comprising cutting a second portion of the sealing of the wound to create a second gap configured to fit a second drain tube of the bandage kit, and simultaneously inserting the drain tube and the second drain tube into the gap and the second gap, respectively, wherein the drain tube and the second drain tube are both attached to the connecting tube.
